(19) United States
(12) Patent Application Publication
(10) Pub. No.: US 2025/0064392 A1
(43) Pub. Date: Feb. 27, 2025

(54) METHOD FOR DETECTING IMPAIRMENT STATUS ON INSTRUMENTAL ACTIVITIES OF DAILY LIVING

(71) Applicant: Linus Health, Inc., Boston, MA (US)

(72) Inventors: Joyce Gomes-Osman, Boston, MA (US); Ali Jannati, Boston, MA (US); Claudio Toro-Serey, Boston, MA (US); Marissa Ciesla, Boston, MA (US); Russell Banks, Boston, MA (US); John Showalter, Boston, MA (US); Sean Tobyne, Boston, MA (US); Alvaro Pascual-Leone, Boston, MA (US); David Bates, Boston, MA (US)

(21) Appl. No.: 18/774,469

(22) Filed: Jul. 16, 2024

Related U.S. Application Data
(60) Provisional application No. 63/533,762, filed on Aug. 21, 2023.

(51) Int. Cl. A61B 5/00; A61B 5/11; G16H 10/20

(52) U.S. Cl. CPC A61B 5/4842; A61B 5/1124; A61B 5/4088; A61B 5/4803; G16H 10/20

(57) ABSTRACT

Systems and methods are disclosed for predicting a functional impairment status. A method for predicting a functional impairment status comprises administering a battery of assessments to a patient, and receiving a plurality of response thereto; collecting performance parameters based on the patient's performance on the battery of assessments; providing the collected performance parameters and the plurality of responses to a pretrained machine learning model; predicting, using the pretrained machine learning model, a likelihood of the patient having an impairment in daily activity; determining a functional impairment status of the patient based on the prediction, and outputting the functional impairment status.

### RELATED APPLICATION(S)

This application claims the benefit of priority to U.S. Provisional Application No. 63/533,762, filed Aug. 21, 2023, which is incorporated herein by reference in its entirety.

### TECHNICAL FIELD

The invention relates generally to a method of detecting a cognitive impairment status, and, in particular, to systems and methods for predicting a patient's ability to perform instrumental activities of daily life using results from cognitive, motor, and lifestyle assessments.

### BACKGROUND

Nearly 6.7 million people aged 65 and older in the US live with Alzheimer's disease (AD). Early detection of dementia presents a difficult challenge, as the earliest signs of cognitive decline often go undetected due to a lack of proactive cognitive and functional assessments. Evaluating the patient's dependence in activities of daily living is a critical factor in detecting progression toward dementia.

The present disclosure describes a proposed system and methods for predicting an individual's functional dependence during activities of daily life in older adults. This prediction method includes the analysis of components of the Linus Health Core Cognitive Evaluation, including (1) the DCTclock™, an FDA-listed Class II medical device, (2) a delayed 3-word verbal memory test, (3) analysis of temporal speech and acoustic voice features, and (4) self-reported survey questions that capture key lifestyle and health factors for cognitive impairment/dementia.

### SUMMARY

According to certain aspects of the present disclosure, systems and methods are disclosed for predicting degrees of functional impairment.

In one embodiment, a method for detecting a functional impairment status comprises administering a battery of assessments to a patient, and receiving a plurality of response thereto; collecting performance parameters based on the patient's performance on the battery of assessments; providing the collected performance parameters and the plurality of responses to a pretrained machine learning model; predicting, using the pretrained machine learning model, a likelihood of the patient having an impairment in daily activity; determining a functional impairment status of the patient based on the prediction, and outputting the functional impairment status.

In some embodiments, the regression model is a random forest classifier.

In some embodiments, the method further comprises determining one or more patient interventions based on the functional impairment status; and implementing the one or more patient interventions.

In some embodiments, the battery of assessments includes at least a DCR assessment, a lifestyle and health questionnaire, and/or a functional activity questionnaire.

In some embodiments, the method further comprises receiving a response to the DCR assessment within the battery of assessments, wherein the response comprises a plurality of individual metrics; combining the host of individual metrics into at least one composite score; aggregating the at least one composite score to determine a final DCR score; and combining the final DCR score with the collected performance parameters and the plurality of responses.

In some embodiments, at least one composite score includes one or more of an information processing score, a drawing efficiency score, a simple and complex motor function score, and a spatial reasoning score.

In some embodiments, a weight for each of the at least one composite score is determined by an ability of the at least one composite score to predict cognitive impairment.

In some embodiments, the lifestyle and health questionnaire comprises at least one question indicating a functional impairment status.

In some embodiments, the method further comprises using a response to the lifestyle and health questionnaire to derive an insight at a group level, wherein a group is based on patient age.

In some embodiments, the method comprises using a response to the lifestyle and health questionnaire to derive an insight at an individual level.

In some embodiments, the functional impairment status is one of mild or moderate.

In some embodiments, the collected performance parameters comprise at least one of a response to a questionnaire, a geometry of a drawing, a stylus derived metric, and/or a speech feature.

In some embodiments, the stylus derived metric includes a force measurement and a directional measurement.

In some embodiments, the speech feature comprises a recording of the patient speaking.

In some embodiments, collecting performance parameters comprises collecting data from at least a touchscreen, a microphone, a webcam, and/or a stylus.

In some embodiments, the method further comprises determining a diagnosis, based on the functional impairment status of the patient.

In some embodiments, the method further comprises determining a therapy, based on the functional impairment status of the patient.

In an embodiment, a system for predicting an impairment status comprises a computing node comprising a computer readable storage medium having program instructions embodied therewith, the program instructions executable by a processor of the computing node to cause the processor to perform a method comprising administering a battery of assessments to a patient, and receiving a plurality of response thereto; collecting performance parameters based on the patient's performance on the battery of assessments; providing the collected performance parameters and the plurality of responses to a pretrained machine learning model; predicting, using the pretrained machine learning model, a likelihood of the patient having an impairment in daily activity; determining a functional impairment status of the patient based on the prediction, and outputting the functional impairment status.

In an embodiment, a computer program product for predicting an impairment status, the computer program product comprising a computer readable storage medium having program instructions embodied therewith, the program instructions executable by a processor to cause the processor to perform a method comprising administering a battery of assessments to a patient, and receiving a plurality of response thereto; collecting performance parameters based on the patient's performance on the battery of assessments; providing the collected performance parameters and the plurality of responses to a pretrained machine learning model; predicting, using the pretrained machine learning model, a likelihood of the patient having an impairment in daily activity; determining a functional impairment status of the patient based on the prediction, and outputting the cognitive impairment status.

In another embodiment, eye-gaze capture is included as an additional modality to understand neurocognitive function, motor function, attentiveness and/or the presence of environmental distractions.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1 is a flow diagram illustrating an exemplary technique for predicting functional performance, in accordance with one or more embodiments of this disclosure.

FIG. 2 is a diagram illustrating an exemplary input and output of a model used to predict a functional performance, in accordance with one or more embodiments of this disclosure.

FIG. 3 is an exemplary computing node.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Ability to perform instrumental activities of daily living (iADL) is typically determined in clinical and rehabilitation settings using paper and pencil cognitive screening tests, and everyday functioning tests, some of which are performance-based and others that are based on information provided by an informant (mostly often the caregiver, family member or sometimes a healthcare provider).

Drawbacks to the current solutions include the requisite use of substantial resources and skilled personnel effort, lack of universal availability, and long completion times. Another drawback of current known solutions is that they rely on information from an informant (caregiver, family member, or sometimes a healthcare provider). There is a need for an inexpensive, universally applicable, and quick solution. A mobile solution would additionally open possibilities for more generalized and widely available screening. Currently, there are universal methods to capture the health, lifestyle and psychosocial factors that are collected by the Life and Health Questionnaire (LHQ). This information is typically captured on paper forms and/or electronic medical records, depending on the test administrator and provider.

Linus Health Digital clock employs Al-enabled analyses of the cognitive assessment completion process to evaluate cognitive and motor function. The Digital clock and Recall test consists of DCTclock™ and a brief assessment of memory including a delayed recall of three words. Four DCTclock™ composite scales including Spatial Reasoning, Drawing Efficiency, Information Processing, and Motor Skills, 44 DCTclock™ subscales (22 from each of Command and Copy drawing components), and the 3-word Delayed Recall score are extracted from the DCR. The LHQ is a brief digital survey that captures key lifestyle and health factors for cognitive impairment/dementia. The combination of the DCR and LHQ together make up the Core Cognitive Evaluation (CCE).

The present disclosure describes a technique for predicting iADLs. These activities include, but are not limited to, cooking, cleaning, transportation, laundry, managing finances, etc., and are evaluated using a digital cognitive assessment technology without the need of an informant. Embodiments of the present disclosure provide a quick, inexpensive, digital, noninvasive solution for detecting a person's level of everyday functioning abilities from an FDA-registered Class II medical device in the DCTclock™, a delayed 3-word recall test and the LHQ. Additional temporal and acoustic voice analysis are conducted on recorded audio, rendering this prediction model a multimodal analysis of graphomotor behavior (DCTclock™), delayed verbal recall, and voice/speech features (spoken recall audio analysis) method.

Using stylus-derived metrics related to motor skills, drawing efficiency, information processing, and spatial reasoning composite scales provided by the DCTclock (detailed below), delayed recall performance, and self-reported information collected on the LHQ, embodiments of the present disclosure detect iADLs in older adults from the Functional Activities Questionnaire (FAQ) or similar instrument, a traditional functional impairment questionnaire completed by an informant aware of the level of functional impairment of the person in question. The described method also uses the questions from the LHQ to detect the functional impairment status group to which users belong without having to take the time for families and caregivers to respond to a functional activities questionnaire. Embodiments of the present disclosure have the ability to use the aforementioned metrics to detect risk of functional impairment that is related to cognitive decline, mild cognitive impairment, and Alzheimer's Disease Related Dementia (ADRD). Embodiments of the present disclosure are delivered on a digital platform and automatically scored, reducing the need for scarce resources and skilled personnel effort. This also allows embodiments of the disclosure to be universally available to anyone with digital access to the test platform, and takes substantially less time to complete than a traditional assessment. Another advantage of the proposed technique is that it does not rely on information obtained from an informant. Accordingly, embodiments of the present disclosure reduce the need for additional personnel needed to complete iADL screening and can indicate those at higher risk for dependence during iADLs.

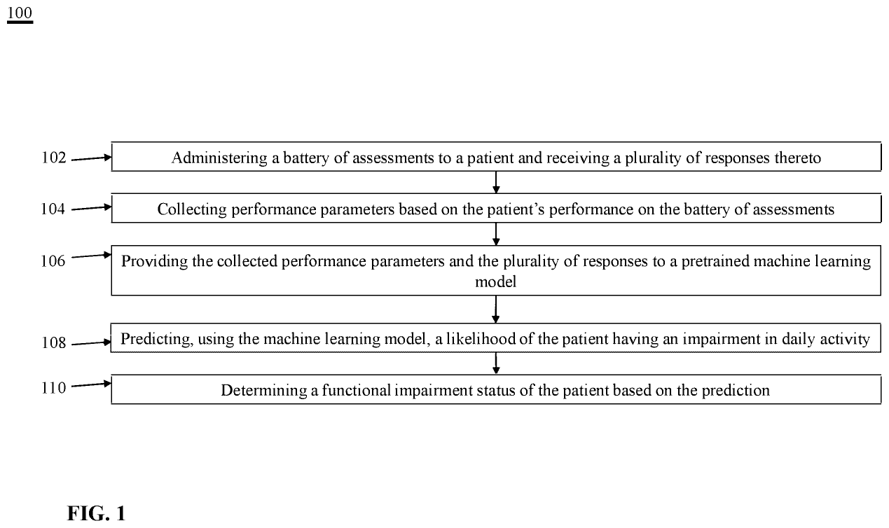

FIG. 1 is a block flow diagram illustrating an exemplary method for detecting the functional impairment status of a user. The exemplary method 100 (e.g., steps 102-110) for predicting the status follows a general workflow process as follows:
A battery of assessments may be administered to a patient subject (step 102), where the battery of assessments may include a DCR assessment, a lifestyle and health questionnaire, and a speech analysis assessment;
Collecting data based on one or more responses to the battery of assessments from the patient (step 104);
Providing the collected data to a machine learning model (step 106), where the machine learning model is a regression model or similar model;
Predicting, using the machine learning model, a likelihood of the patient having an impairment in daily activity (step 108); and
Determining a functional impairment status of the patient based on the prediction (step 110), the determination made by thresholding the output prediction value from the machine learning model. The functional impairment status can be mild functional impairment (e.g., where the score of the prediction is greater than or equal to 6 on the FAQ) or moderate functional impairment (e.g., where the score of the prediction is greater than or equal to 9 on the FAQ).

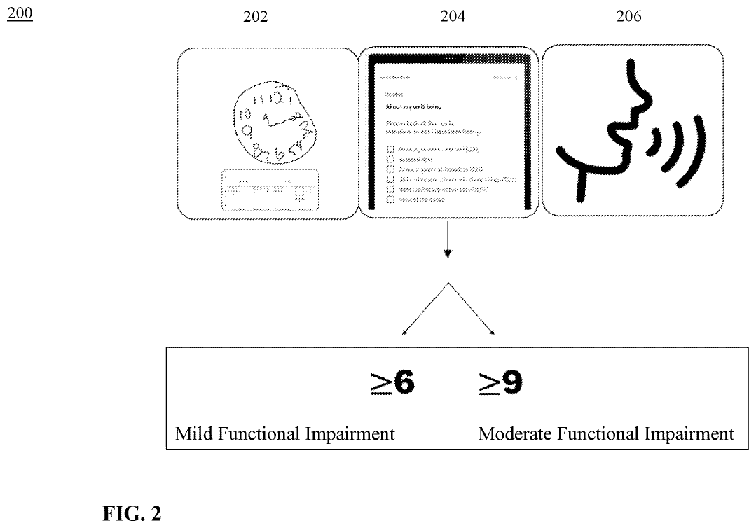

FIG. 2 is a diagram illustrating an exemplary input and output of a model used to predict a functional impairment. Inputs from the DCR (DCTclock drawings and its subscales+delayed recall accuracy) and patient reported responses on the LHQ are used to predict functional performance on the Functional Activities Questionnaire.

After the user completes the CCE, the raw data is analyzed and scoring metrics are produced describing the users performance during the assessment quantitatively. The DCTclock component comprises a host of individual metrics with standard normative values, each related to different aspects of graphomotor and cognitive performance. These subscales are combined into four composite scores (information processing, drawing efficiency, simple and complex motor functions, spatial reasoning), which are then aggregated to create a final score. The influence (or weight) of the subscales and composites is defined by their ability to predict cognitive impairment, and grouped according to guidance from neuropsychologists. Classification based on the final DCT score is then additively combined with delayed recall to provide a DCR score. Answers from the LHQ, which compose the second half of the CCE, are evaluated independently in order to produce risk estimates, and are weighted based on expert advice. Given the neuropsychologically-relevant nature of these components, various groupings of subscales and questions can be extracted to interpret the process of conducting a neuropsychological and/or behavioral task. Differences in these composites and subscales can be associated with functional impairment that could be indicative of cognitive impairment, ADRD or another type of brain-related disability. In addition, changes in these composites and subscales could be predictive of an individual change in functional status that could be indicative of cognitive decline, ADRD or another type of brain-related disability. The LHQ questions quantify key factors that confer risk or protection related with the development of functional decline and ADRD. Specific questions identified can be evaluated for their association with functional impairment that could be indicative of cognitive decline, ADRD or another type of brain-related disability. This information can be used to derive insights at the group level or individual level that could inform clinical decision-making, either in the diagnostic or therapeutic context. At the group level, this information can inform clinicians or other administrators how much more likely users are to face functional deficits than a typical age-matched individual based on their performance on specific metrics. At the individual level, the information can allow the clinician or administrators to predict specific scores for traditional tests that practitioners know well, or estimate the probability of functional deficits based on such estimates.

A multistate study used 941 participants (with age mean±SD of 72±6.7; 57% female; years of education mean±SD=16±2.71; primary language English), classified as cognitively unimpaired (n=402), MCI (n=297), or probable Alzheimer's disease related dementia (n=239). The Functional Activities Questionnaire (FAQ) was the dependent variable that indicated functional impairment, where functional impairment was defined as an FAQ greater or equal to 6. This work was divided into two general steps: 1) examining the association between the summary DCR score and functional impairment, and 2) classifying functional impairment using multimodal features from the DCR. The first goal was examined via adjusted risk ratios (ARR) derived from logistic regressions. The first logistic regression explored differences in functional impairment between individuals with DCR score=0-1 (Red) vs. 4-5 (Green). A second logistic regression explored these differences between individuals with a DCR score of 0 vs. 5. Both logistic regressions included regressors for sex, age, and years of education. The first logistic regression showed that individuals in the Red category were five times more likely to be classified as impaired than those in the Green (ARR=5.10, p<0.0001). Participants in the Yellow category were twice as likely to be classified as impaired than those in Green (ARR=2.12, p<0.05). Age and sex were the only significant demographic predictors (ARR=1.05 and ARR=1.42 for Age and Males, respectively; both p's<0.05), suggesting that older individuals and males were more likely to be classified as functionally impaired. A similar logistic regression comparing individuals with DCR scores of 0 and 5 (n=329) showed that those with a DCR score of 0 were 17 times more likely to demonstrate functional impairment (ARR=17.59, p<0.0001; no significant demographic predictors).

For the second goal, a machine-learning approach was employed using random forests to classify functional impairment. First, participants are split into training (80%) and test (20%) sets, ensuring similar rates of functional impairment across sets (n impaired: train set=25%, test set=27%). Predictors included the DCR-based multimodal drawing, speech, and LHQ features with less than 15% of missing data, as well as age. Missing predictor data on both training and test sets were imputed using the medians from the training set. For the classifier, recursive feature eliminations were performed on 10-fold cross-validated random forests (5 repetitions) on the training set to identify the best model and feature set. Model performance was evaluated using traditional classification metrics (i.e. sensitivity, specificity, negative predictive value, positive predictive value, area under the receiver operating characteristic curve [AUC], and accuracy) on the held-out test set. These metrics were computed on two classification outputs: a binary classification and a three-class classification format. The binary classification was simply the predicted level of impairment by the ML algorithm (impaired vs. unimpaired), with metrics based on optimized decision thresholds provided by the Youden statistic (i.e., the highest balance between sensitivity and specificity). For the three-class format, a pair of thresholded predicted probabilities were determined that would contain a new “Indeterminate” class. For each combination, the resulting indeterminates were removed and calculated performance on the impaired and unimpaired cohorts. The threshold pair that maximized the AUC first and Youden statistic second were selected, as long as the percentage of lost test data was below 25%. Finally, a demographics model was fit to compare as a baseline reference. This model included age, sex, years of education, race, and ethnicity as predictors, and followed the same random forest procedures outlined above. Classification model results were evaluated in two output formats: a binary output (impaired vs. unimpaired) and a three-class output (impaired, indeterminates, and unimpaired). Recursive feature elimination among 2000 multimodal features resulted in a subset of 500 multimodal predictors.

FIG. 2 displays the receiver operating characteristic curves (ROCs) for all model outputs on the held-out test set, indicating that both two- and three-class classification outputs performed better than demographic information alone in classifying functional impairment. A total of 53 participants (24.6%) were classified as indeterminates. Even though the two-class output already showed a strong ability to classify functional impairment, the three-class model improved upon most performance metrics, giving a higher Youden statistic (0.54), accuracy (0.80), and AUC (0.82). Specificity (0.83) and NPV (0.88) were especially high, suggesting that DCR is particularly helpful in ruling out functional impairment and thereby identifying individuals who would not require formal iADL testing.

Indeterminates from the three-class model included 24% of the true positives and 25% of the true negatives from the held-out set. A Poisson regression assessing the interaction in FAQ score differences between each demographic group and the DCR predictions (unimpaired and impaired only, n=162) resulted in significant results only for Whites, whereby White individuals had smaller differences between the predicted impairment classes (z-value=−2.66, p<0.01). This regression also resulted in the main effects of predicted class (z-value=2.77, p<0.001), Race (z-value=2.89, p<0.01), and Sex (z-value=3.78, p<0.001), indicating observed differences in FAQ scores between these demographic groups. Finally, when looking at performance metrics in the test set per demographics, all demographic groups except for Whites showed high specificity (>0.75), and all groups achieved high NPV (>0.82). These numbers match the overall classification performance trends from the full test set. While the interactions in the Poisson regression should be interpreted with care due to the low number of observations, these results support the conclusion that predictions of functional impairment by the DCR are similar across demographic groups.

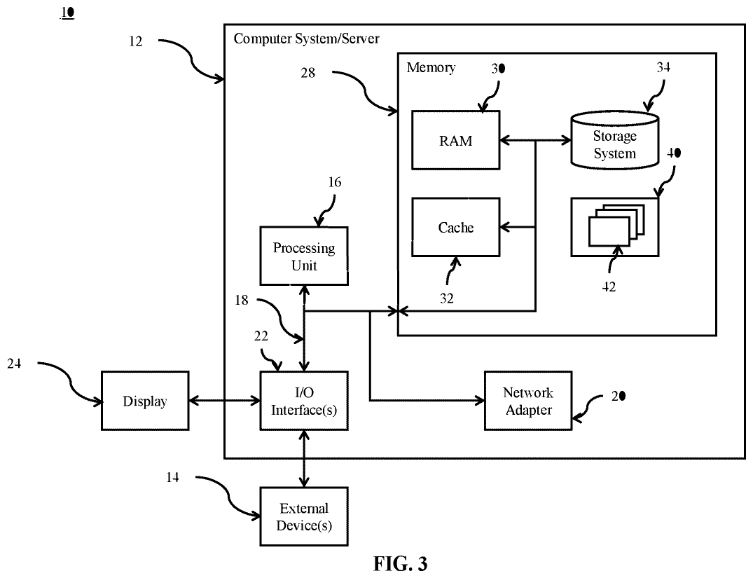

Referring now to FIG. 3, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 3, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a hard drive). Although not shown, a magnetic disk drive for reading from and writing to a removable, non-volatile magnetic disk (e.g., a floppy disk), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non exhaustive list of more specific examples of the computer readable storage medium includes the following: a portable computer diskette, a hard disk, a random access memory (RAM), a read-only memory (ROM), an erasable programmable read-only memory (EEPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

TABLE 1
Feature definitions.

| Feature | Definition |
|---|---|
| Delayed Recall | The accuracy count of the 3-word recall test |
| accuracy score | displayed in a score of 0-3. |
| DCTclock Spatial | The spatial abilities demonstrated during the |
| Reasoning | process of drawing a copy of the displayed |
| composite score | clock. This feature considers metrics |
| | pertaining to the geometric properties of the |
| | drawing including the circularity of the |
| | clock circle, placement of clock |
| | components, and drawing placement on the |
| | page. |
| DCTclock Oscillatory | A measure of how much the motion of the |
| Motion | pen deviates from a smooth pen motion |
| | during the drawing process of the copying |
| | of the clock. |
| Stylus Objective Feature: | The force of the touch, where a value of 1 |
| Pressure | represents the force of an average touch, a |
| | predetermined benchmark set by the stylus |
| | system. The force reported is measured |
| | along the axis of the pencil and is included |
| | into a deep learning model that produces a |
| | feature prediction for each patient. |
| Objective Feature: Azimuth | A measure of the direction in which the |
| | stylus is pointing in radians relative to the |
| | plane of the screen. The azimuth angle |
| | increases as the user swings the cap-end |
| | of the stylus in a clockwise direction |
| | around the tip included into a deep |
| | learning model that produces a feature |
| | prediction for each patient. |
| Objective Feature: Altitude | A measure of the elevation of the stylus |
| | above the plane of the screen in radians. A |
| | value of 0 radians indicates that the stylus |
| | is parallel to the surface, which increases |
| | as the user lifts the cap-end from the |
| | surface and is included into a deep |
| | learning model that produces a feature |
| | prediction for each patient. |
| Objective Feature: | The number of individual pen strokes |
| Stroke Count | produced in the drawing of either the |
| | command or copy clock, with each stroke |
| | delineated by each stylus point-end lift from |
| | the tablet surface and incorporated into a |
| | deep learning model that produces a feature |
| | prediction for each patient. |
| Linus Life and Health | The Life and Health Questionnaire (LHQ) |
| Questionnaire: | is a brief digital survey that captures key |
| Subjective/Patient-reported | lifestyle and health factors for cognitive |
| Depression/Anxiety | impairment/dementia. These questions have |
| Frailty | been demonstrated to significantly predict |
| Sleep | functional status both in isolation and also |
| Nutrition | when combined with other metrics listed |
| Physical Activity | above. |
| Social engagement | |
| Cognitive stimulation | |
| Life Satisfaction/Purpose | |

What is claimed:

1. A method for predicting a functional impairment status, comprising:
administering a battery of assessments to a patient, and receiving a plurality of response thereto;
collecting performance parameters based on the patient's performance on the battery of assessments;
providing the collected performance parameters and the plurality of responses to a pretrained machine learning model;
predicting, using the pretrained machine learning model, a likelihood of the patient having an impairment in daily activity;
determining a functional impairment status of the patient based on the prediction, and
outputting the functional impairment status.

2. The method of claim 1, wherein the machine learning model is a random forest classifier.

3. The method of claim 1, further comprising:
determining one or more patient interventions based on the functional impairment status; and
implementing the one or more patient interventions.

4. The method of claim 1, wherein the battery of assessments includes at least a DCR assessment, a lifestyle and health questionnaire, and/or a functional activity questionnaire.

5. The method of claim 5, further comprising:
receiving a response to the DCR assessment within the battery of assessments, wherein the response comprises a plurality of individual metrics;
combining the host of individual metrics into at least one composite score;
aggregating the at least one composite score to determine a final DCR score; and
combining the final DCR score with the collected performance parameters and the plurality of responses.

6. The method of claim 6, wherein the at least one composite score includes one or more of an information processing score, a drawing efficiency score, a simple and complex motor function score, and a spatial reasoning score.

7. The method of claim 6, wherein a weight for each of the at least one composite score is determined by an ability of the at least one composite score to predict cognitive impairment.

8. The method of claim 5, wherein the lifestyle and health questionnaire comprises at least one question indicating a functional impairment status.

9. The method of claim 9, further comprising using a response to the lifestyle and health questionnaire to derive an insight at a group level, wherein a group is based on patient age.

10. The method of claim 9, further comprising using a response to the lifestyle and health questionnaire to derive an insight at an individual level.

11. The method of claim 1, wherein the functional impairment status is one of mild or moderate.

12. The method of claim 1, wherein the collected performance parameters comprise at least one of a response to a questionnaire, a geometry of a drawing, a stylus derived metric, and/or a speech feature.

13. The method of claim 13, wherein the stylus derived metric includes a force measurement and a directional measurement.

14. The method of claim 13, wherein the speech feature comprises a recording of the patient speaking.

15. The method of claim 1, wherein collecting performance parameters comprises collecting data from at least a touchscreen, a microphone, a webcam, and/or a stylus.

16. The method of claim 1, further comprising determining a diagnosis, based on the functional impairment status of the patient.

17. The method of claim 1, further comprising determining a therapy, based on the functional impairment status of the patient.

18. A system for predicting an impairment status, the system comprising:
a computing node comprising a computer readable storage medium having program instructions embodied therewith, the program instructions executable by a processor of the computing node to cause the processor to perform a method comprising:
administering a battery of assessments to a patient, and receiving a plurality of response thereto;
collecting performance parameters based on the patient's performance on the battery of assessments;
providing the collected performance parameters and the plurality of responses to a pretrained machine learning model;
predicting, using the pretrained machine learning model, a likelihood of the patient having an impairment in daily activity;
determining a functional impairment status of the patient based on the prediction, and
outputting the cognitive impairment status.

19. A computer program product for predicting an impairment status, the computer program product comprising a computer readable storage medium having program instructions embodied therewith, the program instructions executable by a processor to cause the processor to perform a method comprising:
administering a battery of assessments to a patient, and receiving a plurality of response thereto;
collecting performance parameters based on the patient's performance on the battery of assessments;
providing the collected performance parameters and the plurality of responses to a pretrained machine learning model;
predicting, using the pretrained machine learning model, a likelihood of the patient having an impairment in daily activity;
determining a functional impairment status of the patient based on the prediction, and outputting the cognitive impairment status.
